(19) United States
(12) Patent Application Publication
(10) Pub. No.: US 2024/0286983 A1
(43) Pub. Date: Aug. 29, 2024

(54) PREPARATION SYSTEMS AND PREPARATION METHODS FOR XYLITOL CRYSTALS

(71) Applicant: ZHEJIANG HUAKANG PHARMACEUTICAL CO., LTD., Quzhou (CN)

(72) Inventors: Qiang WU, Quzhou (CN); Mian LI, Quzhou (CN); Wulong YANG, Quzhou (CN); Fuan WAN, Quzhou (CN); Weidong XU, Quzhou (CN); Shufang QIN, Quzhou (CN); Hang YUE, Quzhou (CN); Weijia WANG, Quzhou (CN)

(73) Assignee: ZHEJIANG HUAKANG PHARMACEUTICAL CO., LTD., Quzhou (CN)

(21) Appl. No.: 18/656,576

(22) Filed: May 6, 2024

Related U.S. Application Data
(63) Continuation-in-part of application No. PCT/CN2023/096364, filed on May 25, 2023.

(30) Foreign Application Priority Data
Dec. 7, 2022 (CN) 202211567511.2

(51) Int. Cl. C07C 29/78; B01D 9/00; B01D 15/36; B01D 21/26; B01D 61/02; B01D 69/02; C07C 29/76

(52) U.S. Cl. CPC C07C 29/78; B01D 9/0022; B01D 9/0036; B01D 9/0063; B01D 15/361; B01D 21/262; B01D 61/027; B01D 69/02; C07C 29/76; B01D 2009/0086; B01D 2325/34

(57) ABSTRACT

The present disclosure provides a preparation system and a preparation method for xylitol crystals. In the preparation system, outlets of a first centrifuge are connected with an inlet of a hot air drying tank and an inlet of a primary mother liquor storage tank through pipelines, respectively. Outlets of a second centrifuge are connected with an inlet of a second fluidized bed dryer and an inlet of a secondary mother liquor storage tank through pipelines, respectively. An outlet of a dicrystalline sugar dissolution tank is connected with an inlet of a blending tank through pipeline. An outlet of a tricrystalline sugar dissolution tank is connected with the inlet of the primary mother liquor storage tank. The blending tank is provided with an inlet for a raw material of xylitol hydrogenation solution. An output of an outlet of a first fluidized bed dryer is prepared xylitol crystals.

### CROSS-REFERENCE TO RELATED APPLICATIONS

This application is a continuation-in-part of International Application No. PCT/CN2023/096364, filed on May 25, 2023, which claims the priority of Chinese application No. 202211567511.2, filed on Dec. 7, 2022, the entire contents of each of which are incorporated herein by reference.

### TECHNICAL FIELD

The present disclosure relates to the field of xylitol preparation, and in particular to a preparation system and a preparation method for xylitol crystals.

### BACKGROUND

The existing xylitol production process uses corn kernels as raw materials to obtain xylitol crystals through hydrolysis, hydrogenation, decolorization, ion exchange, concentration, crystallization, centrifugation, drying, etc. The crystallization determines the quality and yield of xylitol products, which plays a decisive role in the morphology, purity, bulk density, particle size distribution, and caking properties of the crystal products.

The thrust of the xylitol crystallization process mainly comes from the thermodynamically non-equilibrium properties of the crystallization system. Solubility, mesa-stable region, induction period, and other crystallization thermodynamic properties have a great impact on the selection of crystallization method and crystallization yield. At present, the crystallization method commonly used in industry includes evaporation crystallization and cooling crystallization. Both evaporation crystallization and cooling crystallization are well applied to the crystallization process of xylitol due to the large solubility of xylitol.

However, as for evaporation crystallization or cooling crystallization, due to unreasonable process parameters, or the lack of theoretical and factual basis for the time of adding crystal seeds or adding the crystal seeds based on experience, problems such as time-consuming during xylitol crystallization process, severe secondary nucleation, high fine powder content, significant product variations between different batches, and unstable crystallization process, which leads to caking of xylitol products.

Therefore, from the perspective of industrial implementation, optimizing the crystallization process parameters to reduce the phenomenon of secondary nucleation and control the particle size distribution of the product has become one of the most important measures to avoid or alleviate the caking of the xylitol crystals.

### SUMMARY

One of the embodiments of the present disclosure provides a preparation system for xylitol crystals. The preparation system may comprise a blending tank, a decolorization tank, an ion exchange column, a nanofiltration system, a first evaporator, a first crystallization kettle, a first centrifuge, a hot air drying tank, and a first fluidized bed dryer that are sequentially connected through pipelines; a primary mother liquor storage tank, a second evaporator, a second crystallization kettle, a second centrifuge, a second fluidized bed dryer, and a dicrystalline sugar dissolution tank that are sequentially connected through pipelines; and a secondary mother liquor storage tank, a third evaporator, a third crystallization kettle, a third centrifuge, a third fluidized bed dryer, and a tricrystalline sugar dissolution tank that are sequentially connected through pipelines. A solid outlet of the first centrifuge may be connected with an inlet of the hot air drying tank through pipeline. A liquid outlet of the first centrifuge may be connected with an inlet of the primary mother liquor storage tank through pipeline. A solid outlet of the second centrifuge may be connected with an inlet of the second fluidized bed dryer through pipeline. A liquid outlet of the second centrifuge may be connected with an inlet of the secondary mother liquor storage tank through pipeline. The dicrystalline sugar dissolution tank may be provided with a water inlet for pure water. An outlet of the dicrystalline sugar dissolution tank may be connected with an inlet of the blending tank. The tricrystalline sugar dissolution tank may also be provided with a water inlet for pure water. An outlet of the tricrystalline sugar dissolution tank may be connected with an inlet of the primary mother liquor storage tank through pipeline. The blending tank may be provided with an inlet for a raw material of xylitol hydrogenation solution. The blending tank may be used for mixing the raw material of xylitol hydrogenation solution with dicrystalline sugar solution delivered from the dicrystalline sugar dissolution tank. An output material of an outlet of the first fluidized bed dryer may be prepared xylitol crystals.

One of the embodiments of the present disclosure further provides a preparation method for xylitol crystals. The preparation method may be performed using the preparation system for the xylitol crystals. The preparation method may comprise: operation 210, in a blending tank, blending the raw material of xylitol hydrogenation solution with the dicrystalline sugar solution in a certain ratio, and then sequentially passing mixed solution including the raw material of xylitol hydrogenation solution blended with the dicrystalline sugar solution through the decolorization tank for decolorization treatment, the ion exchange column for impurity removal treatment, the nanofiltration system for filtration treatment, and the first evaporator for evaporation and concentration treatment to obtain xylitol concentrate solution, wherein brix of the xylitol concentrate solution may be within a range of 78-82%, temperature of the xylitol concentrate solution may be within a range of 90-100° C., and conductivity of the xylitol concentrate solution may be smaller than 20 μs/cm; operation 220, entering the xylitol concentrate solution into the first crystallization kettle through pipeline, including: feeding the first crystallization kettle with a first volume of xylitol concentrate solution, controlling a vacuum degree of the first crystallization kettle to be within a range from −0.095 MPa to −0.098 MPa and temperature of the first crystallization kettle to be within a range from 64° C. to 68° C., and adding xylitol crystal seeds until xylitol crystals begin to crystalize in the first crystallization kettle and a count of particles of xylitol crystals observed from a view window of the first crystallization kettle is within a preset range, adding a second volume of xylitol concentrate solution to the first crystallization kettle, performing a variable frequency stirring for 7-12 h, and at an end of crystallization, feeding steam to increase system temperature by 1-2° C. and maintaining the system temperature for 0.5-2.0 h to obtain xylitol sugar paste, wherein a ratio of the first volume to the second volume may be within a range of 1:0.8-1.5; operation 230, using the first centrifuge to separate the xylitol sugar paste obtained in operation 220 to obtain crystalline xylitol and primary mother liquor, entering the primary mother liquor into the primary mother liquor tank through pipeline for temporary storage, and passing the crystalline xylitol through the hot air drying tank for drying treatment and the first fluidized bed dryer for cold air drying treatment to obtain the prepared xylitol crystals; sequentially passing the primary mother liquor through the second evaporator for concentration treatment, the second crystallization kettle for crystallization treatment, and the second centrifuge for centrifugation treatment to obtain dicrystalline sugar and secondary mother liquor, and entering the secondary mother liquor into the secondary mother liquor tank through pipeline for temporary storage; passing the dicrystalline sugar through the second fluidized bed dryer for drying treatment and the dicrystalline sugar dissolution tank for dissolution treatment, inputting the pure water to the dicrystalline sugar dissolution tank to dissolve the dicrystalline sugar to obtain dicrystalline sugar solution, and entering the dicrystalline sugar solution into the blending tank through pipeline for blending; and operation 240, sequentially passing the secondary mother liquor obtained in operation 230 through the third evaporator for concentration treatment, through the third crystallization kettle for crystallization treatment, and through the third centrifuge for centrifugation treatment to obtain tricrystalline sugar and tertiary mother liquor, passing the tricrystalline sugar through the third fluidized bed dryer for drying treatment and the tricrystalline sugar dissolution tank for dissolution treatment, inputting the pure water to the tricrystalline sugar dissolution tank to dissolve the tricrystalline sugar to obtain tricrystalline sugar solution, and entering the tricrystalline sugar solution into the primary mother liquor tank through pipeline to blend the tricrystalline sugar solution with the primary mother liquor, the tertiary mother liquor being stored as liquid xylitol.

### BRIEF DESCRIPTION OF THE DRAWINGS

The present disclosure is further illustrated by way of exemplary embodiments, which are described in detail with reference to the accompanying drawings. These embodiments are not limiting. In these embodiments, the same numbering indicates the same structure, wherein:

FIG. 1 is a block diagram illustrating an exemplary preparation system for xylitol crystals according to some embodiments of the present disclosure;

FIG. 2 is a flowchart illustrating an exemplary preparation method for xylitol crystals according to some embodiments of the present disclosure;

FIG. 3 is a flowchart illustrating an exemplary process for determining a frequency conversion instruction according to some embodiments of the present disclosure; and

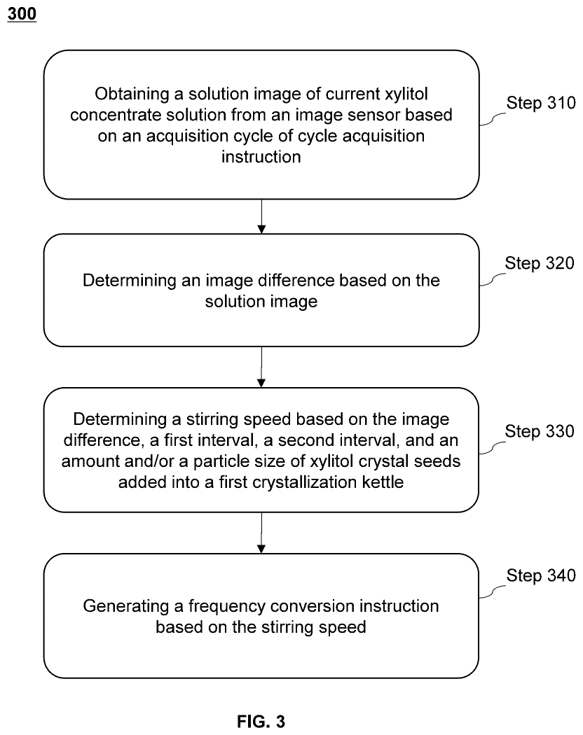

FIG. 4 is a schematic diagram illustrating a particle size distribution of xylitol crystals prepared in some embodiments of the present disclosure and Comparative Examples.

### DETAILED DESCRIPTION

In order to more clearly illustrate the technical solutions of the embodiments of the present disclosure, the following briefly introduces the drawings that need to be used in the description of the embodiments. Apparently, the accompanying drawings in the following description are only some examples or embodiments of the present disclosure, and those skilled in the art can also apply the present disclosure to other similar scenarios according to the drawings without creative efforts. Unless obviously obtained from the context or the context illustrates otherwise, the same numeral in the drawings refers to the same structure or operation.

It should be understood that “system,” “device,” “unit,” and “module” as used herein are methods for distinguishing different components, elements, parts, portions or assemblies of different levels. However, the words may be replaced by other expressions if other words can achieve the same purpose.

As indicated in the disclosure and claims, the terms “a,” “an,” and/or “the” are not specific to the singular form and may include the plural form unless the context clearly indicates an exception. Generally speaking, the terms “comprising” and “including” only suggest the inclusion of clearly identified steps and elements, and these steps and elements do not constitute an exclusive list, and the method or device may also contain other steps or elements.

The flowchart is used in the present disclosure to illustrate the operations performed by the system according to the embodiments of the present disclosure. It should be understood that the preceding or following operations are not necessarily performed in the exact order. Instead, various steps may be processed in reverse order or simultaneously. Meanwhile, other operations may be added to these procedures, or a certain step or steps may be removed from these procedures.

The present disclosure provides a preparation system and a preparation method for xylitol crystals, which enhance a particle size of a xylitol crystal product by optimizing parameters of a crystallization process, reducing the phenomenon of secondary nucleation, and controlling the particle size distribution of the product, thereby prolonging a caking cycle of the xylitol crystal product.

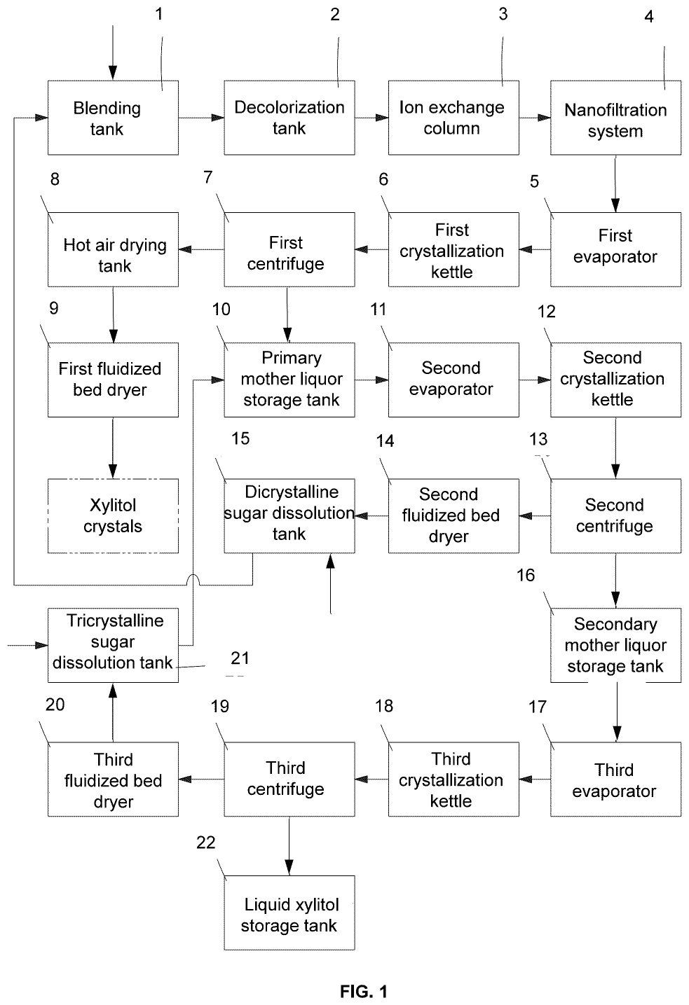

FIG. 1 is a block diagram illustrating an exemplary preparation system for xylitol crystals according to some embodiments of the present disclosure.

As illustrated in FIG. 1, the preparation system for xylitol crystals may comprise a blending tank 1, a decolorization tank 2, an ion exchange column 3, a nanofiltration system 4, a first evaporator 5, a first crystallization kettle 6, a first centrifuge 7, a hot air drying tank 8, and a first fluidized bed dryer 9 that are sequentially connected through pipelines; a primary mother liquor storage tank 10, a second evaporator 11, a second crystallization kettle 12, a second centrifuge 13, a second fluidized bed dryer 14, and a dicrystalline sugar dissolution tank 15 that are sequentially connected through pipelines; and a secondary mother liquor storage tank 16, a third evaporator 17, a third crystallization kettle 18, a third centrifuge 19, a third fluidized bed dryer 20, and a tricrystalline sugar dissolution tank 21 that are sequentially connected through pipelines. Arrows in the figure indicate the material flow direction in the system.

In some embodiments, the solid outlet of the first centrifuge 7 may be connected with inlet of the hot air drying tank 8 through pipeline. the liquid outlet of the first centrifuge 7 may be connected with inlet of the primary mother liquor storage tank 10 through pipeline. the solid outlet of the second centrifuge 13 may be connected with inlet of the second fluidized bed dryer 14 through pipeline. the liquid outlet of the second centrifuge 13 may be connected with inlet of the secondary mother liquor storage tank 16 through pipeline.

In some embodiments, the dicrystalline sugar dissolution tank 15 may be provided with a water inlet for pure water. the outlet of the dicrystalline sugar dissolution tank 15 may be connected with inlet of the blending tank 1 through pipeline. The tricrystalline sugar dissolution tank 21 may also be provided with a water inlet for pure water. the outlet of the tricrystalline sugar dissolution tank 21 may be connected with inlet of the primary mother liquor storage tank 10 through pipeline.

In some embodiments, the blending tank 1 may also be provided with a feed pipe for xylitol hydrogenation solution. The blending tank 1 may be configured to mix raw material of xylitol hydrogenation solution with dicrystalline sugar solution delivered from the dicrystalline sugar dissolution tank 15. The output material of the outlet of the first fluidized bed dryer 9 may be prepared xylitol crystals.

In some embodiments, the blending tank 1 may store the raw material of xylitol hydrogenation solution for preparing xylitol crystals. The xylitol hydrogenation solution may be fed into the blending tank 1 before the preparation system prepares the xylitol crystals, or may be simultaneously fed into the blending tank 1 with the dicrystalline sugar solution delivered from the dicrystalline sugar dissolution tank 15.

In some embodiments, the preparation system may further comprise a liquid xylitol storage tank 22. The outlet of the third centrifuge 19 may be connected with inlet of the liquid xylitol storage tank 22 through pipeline.

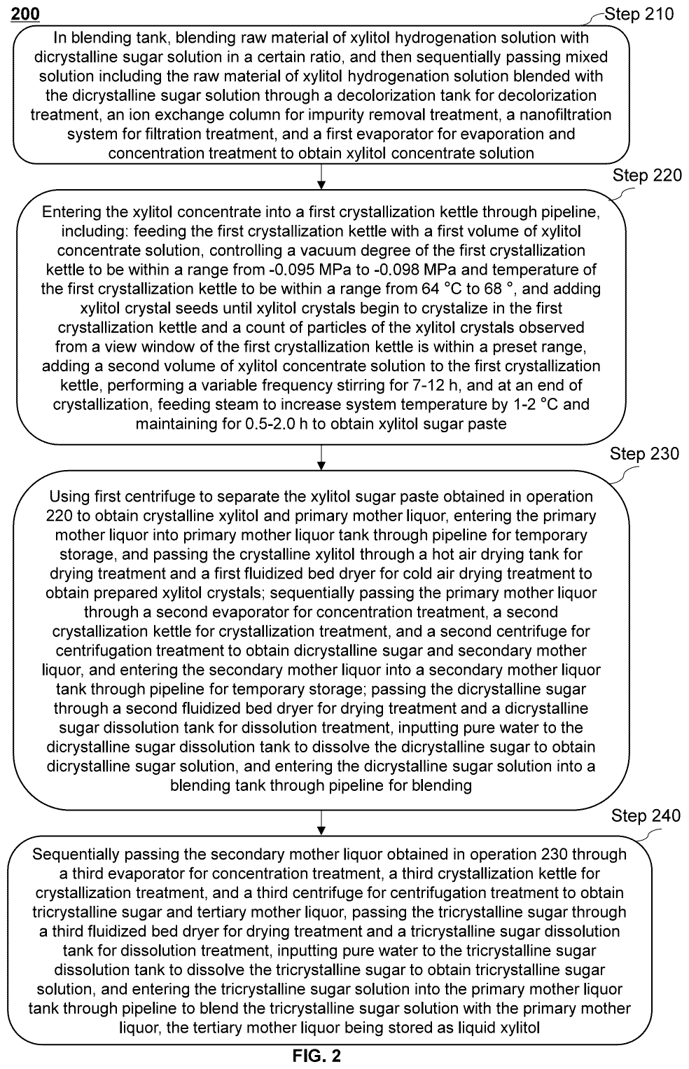

Referring to FIG. 2, the embodiments of the present disclosure further disclose a preparation method for xylitol crystals. One or more operations in a process 200 of the method may be performed using the preparation system for xylitol crystals as illustrated in the aforementioned embodiments. Process 200 may include the following operations.

Operation 210, in blending tank 1, the raw material of xylitol hydrogenation solution may be blended with dicrystalline sugar solution in a certain ratio, and then the mixed solution including the raw material of xylitol hydrogenation solution blended with the dicrystalline sugar solution may be passed through the decolorization tank 2 for decolorization treatment, the ion exchange column 3 for impurity removal treatment, the nanofiltration system for filtration treatment, and the first evaporator 5 for evaporation and concentration treatment to obtain xylitol concentrate solution. The brix of the xylitol concentrate solution may be within a range of 78-82%, the temperature of the xylitol concentrate solution may be within a range of 90-100° C., and the conductivity of the xylitol concentrate solution may be smaller than 20 μs/cm.

Operation 220, the xylitol concentrate solution may enter the first crystallization kettle 6 through pipeline. Specifically, the first crystallization kettle 6 may be first fed with a first volume of xylitol concentrate solution firstly. The vacuum degree of the first crystallization kettle 6 may be controlled to be within a range from −0.095 MPa to −0.098 MPa and the temperature of the first crystallization kettle 6 may be controlled to be within a range from 64° C. to 68° C., and xylitol crystal seeds may be added to stimulate crystallization until xylitol crystals begin to crystallize in the first crystallization kettle 6 and a count of particles of xylitol crystals observed from a view window of the first crystallization kettle is within a preset range. At the same time, a second volume of xylitol concentrate solution may be added to the first crystallization kettle 6, and variable frequency stirring may be performed for 7-12 h. At the end of crystallization, steam may be fed to the first crystallization kettle the increase system temperature by 1-2 ° C. (e.g., control the system temperature to be within a range of 65-70° C.) and maintain for 0.5-2.0 h to obtain a xylitol sugar paste. A ratio of the first volume to the second volume may be within a range of 1:0.8-1.5.

Operation 230, using the first centrifuge 7 to separate the xylitol sugar paste obtained in operation 220 to obtain crystalline xylitol and primary mother liquor. The primary mother liquor may enter the primary mother liquor tank 10 through pipeline for temporary storage. The crystalline xylitol may be passed through the hot air drying tank 8 for drying treatment and the first fluidized bed dryer 9 for cold air drying treatment to obtain a xylitol crystal product (i.e., prepared xylitol crystals).

The primary mother liquor may sequentially pass the second evaporator 11 for concentration treatment, the second crystallization kettle 12 for crystallization treatment, and the second centrifuge 13 for centrifugation treatment to obtain dicrystalline sugar and secondary mother liquor. The secondary mother liquor may enter the secondary mother liquor tank 16 through pipeline for temporary storage. The dicrystalline sugar may pass through the second fluidized bed dryer 14 for drying treatment and then the dicrystalline sugar dissolution tank 15 for dissolution treatment. Pure water may be input into the dicrystalline sugar dissolution tank to dissolve the dicrystalline sugar to obtain dicrystalline sugar solution. The dicrystalline sugar solution may enter the blending tank 1 through pipeline to be blended with raw material of xylitol hydrogenation solution.

Operation 240, the secondary mother liquor obtained in operation 230 may sequentially pass through the third evaporator 17 for concentration treatment, the third crystallization kettle 18 for crystallization treatment, and the third centrifuge 19 for centrifugation treatment to obtain tricrystalline sugar and tertiary mother liquor.

In some embodiments, the tricrystalline sugar may pass through the third fluidized bed dryer 20 for drying treatment and then the tricrystalline sugar dissolution tank 21 for dissolution treatment. The pure water may be input into the tricrystalline sugar dissolution tank to dissolve the tricrystalline sugar to obtain tricrystalline sugar solution. The tricrystalline sugar solution may enter the primary mother liquor tank 10 through pipeline to be blended with primary mother liquor. The tertiary mother liquor may be stored as liquid xylitol.

In some embodiments, the tricrystalline sugar solution may be blended with primary mother liquor for reuse.

In some embodiments, in operation 210, the molecular weight cut-off (MWCO) of nanofiltration membrane of the nanofiltration system 4 may be within a range of 300-800 Da, thereby ensuring a filtration effect, and improving the purity of the xylitol concentrate solution.

In some embodiments, in operation 220, the first crystallization kettle 6 may be a vacuum evaporation crystallization kettle. In some embodiments, to ensure a good crystallization effect, the amount of the xylitol crystal seeds may account 0.0004-0.0008% of the mass of solution in the first crystallization kettle 6, and the particle size of the xylitol crystal seeds may be within a range of 80-100 mesh.

In some embodiments, in operation 220, the xylitol sugar paste may be sprayed and washed for 5-10 s using 80-100% concentration ethanol solution after the separation treatment is performed on the xylitol sugar paste using the first centrifuge 7 to remove water from the crystal xylitol.

In some embodiments, in operation 230, the temperature of the hot air of the hot air drying tank 8 may be within a range of 75-85 ° C. to accelerate the drying speed while ensuring the drying effect.

In some embodiments, in operation 220, the variable frequency stirring refers to that the stirring speed gradually reduces from 90 rpm to 10 rpm to improve the crystallization quality of the xylitol crystals and improve the particle size of the xylitol crystal product.

In some embodiments, the preparation system for the xylitol crystals may further comprise an image sensor and a microprocessor. The microprocessor may be configured to: generate a frequency conversion instruction and send the frequency conversion instruction to the first crystallization kettle to adjust the stirring speed of the first crystallization kettle; and generate a cycle acquisition instruction and send the cycle acquisition instruction to the image sensor.

The image sensor refers to a device capable of obtaining an image of an interior of the first crystallization kettle. For example, the image sensor may be a camera or a video camera. In some embodiments, the image sensor may be configured to obtain a solution image of the xylitol concentrate solution, and further determine, based on the solution image, the count of particles of xylitol crystals in the xylitol concentrate solution and the rate of crystallization.

In some embodiments, the image sensor may be located near the view window of the first crystallization kettle to obtain the image of the interior of the first crystallization kettle. In some embodiments, the size of the view window of the first crystallization kettle may be 1 cm². It should be noted that the size of the view window may vary depending on the size or model of the first crystallization kettle used in the system. For example, the size of the view window may be 0.8 cm² or 5 cm².

In some embodiments, to obtain a clearer image of the interior of the first crystallization kettle, for example, to determine the state of the solution or the count and size of crystals within the first crystallization kettle based on the image of the interior, the image sensor may include a microscope. The microscope enables a clearer observation of the count of the particles of the xylitol crystals and the rate of crystallization through the view window. In some embodiments, the microscope may be a 10× (eyepiece) 10× (objective) magnification microscope, or another magnification microscope such as a 5×100 or 16×40 magnification microscope.

The frequency conversion instruction refers to an electrical signal generated by the microprocessor and sent to the first crystallization kettle for regulating the stirring speed of the first crystallization kettle. For example, the stirring speed of the first crystallization kettle may be regulated by increasing or decreasing the rotation speed of a stirring paddle in the first crystallization kettle.

The cycle acquisition instruction refers to an electrical signal generated by the microprocessor and sent to the image sensor for directing the image sensor to periodically acquire the solution image of the xylitol concentrate solution in the first crystallization kettle. In some embodiments, the acquisition cycle may be manually set. For example, the image sensor may be controlled to acquire the solution image every 5 seconds or every 10 seconds.

By continuously acquiring and analyzing the solution image of the xylitol concentrate solution, the state of the solution can be better analyzed to control the crystallization process of the xylitol concentrate solution.

In some embodiments, the microprocessor may determine the frequency conversion instruction based at least on the acquired solution image to achieve variable frequency stirring in the first crystallization kettle. In some embodiments, the microprocessor may be further configured to perform the following operations.

Operation 310: a solution image of current xylitol concentrate solution may be obtained from the image sensor based on an acquisition cycle of a cycle acquisition instruction.

Operation 320: an image difference may be determined based on the solution image.

The image difference refers to a difference between pixels of two adjacent solution images. In some embodiments, the image difference may be a matrix, and a matrix dimension may be the same as the pixels of the solution image. For example, if the resolution of the solution image is 500×500 pixels, the image difference dimension may be a 500×500 matrix, and each element in the matrix may correspond to the difference between the pixels of the solution images.

Because the morphology of the solution does not change significantly during the xylitol crystallization process, and the crystals appearing in the solution are more clearly visible in the solution image, the difference between the pixels of the two adjacent solution images may mainly reflect the count of the crystals and the rate of crystallization. Therefore, whether the stirring speed needs to be regulated may be determined based on the image difference.

Operation 330, the stirring speed may be determined based on the image difference, a first interval, a second interval, and an amount or a particle size of xylitol crystal seeds added into the first crystallization kettle.

In some embodiments, the first interval refers to a time difference between the time when the xylitol concentrate solution enters the first crystallization kettle and the time when the xylitol crystal seeds are added, and the second interval refers to a time difference between the time when the xylitol crystal seeds are added and the current time.

In some embodiments, the first interval and the second interval may be determined by the actual time difference in operation 220. For example, the time when the xylitol concentrate solution enters the first crystallization kettle and the time when the xylitol crystalline seeds are added may be recorded, and the microprocessor may calculate the time difference between the time when the xylitol concentrate solution enters the first crystallization kettle and the time when the xylitol crystalline seeds are added, as well as the time difference between the time when the xylitol crystalline seeds are added and the current time to obtain the first interval and the second interval, respectively.

The amount and/or the particle size of the xylitol crystal seeds may be manually input based on the actual situation of the preparation method for xylitol crystals. In some embodiments, the amount and/or the particle size of xylitol crystal seeds added into the first crystallization kettle may be derived from the data used in the preparation method mentioned above.

In some embodiments, the microprocessor may determine, based on the image difference, the first interval, the second interval, and the amount and/or the particle size of the added xylitol crystal seeds added into the first crystallization kettle, the stirring speed through vector matching.

In some embodiments, the microprocessor may collect historical data of xylitol crystallization, select a portion of the historical data of which the actual crystallization quality satisfies a requirement, and slice the portion of historical data of which the actual crystallization quality satisfies the requirement based on a predetermined time period (e.g., every 5 minutes) to obtain a plurality of reference historical time periods. Each of the plurality of reference historical time periods may include a historical image difference, a historical first interval, a historical second interval, a historical amount and/or particle size of xylitol crystal seeds added into the first crystallization kettle, and a stirring peed of a next adjacent reference historical time period.

In some embodiments, the microprocessor may cluster the plurality of reference historical time periods using the K-means algorithm. Further, the image difference, the first interval, the second interval, and the amount and/or the particle size of xylitol crystal seeds added into the first crystallization kettle corresponding to clustering centers obtained by clustering may be used as standard vectors to construct a standard vector table.

In some embodiments, in operation 330, the microprocessor may construct a matching vector based on the current image difference, the first interval, the second interval, and the amount and/or the particle size of the xylitol crystal seeds added into the first crystallization kettle; determine a standard vector in the standard vector table that has a highest similarity or a smallest vector distance to the matching vector; and use the stirring speed of the next adjacent reference historical time period corresponding to the standard vector as the stirring speed.

In some embodiments, the stirring speed may be determined through a machine learning model. In some embodiments, the microprocessor may determine the stirring speed based on the image difference, the first interval, the second interval, and the amount and/or the particle size of xylitol crystal seeds added into the first crystallization kettle, through a stirring speed determination model. The stirring speed determination model may be a machine learning model.

In some embodiments, the stirring speed determination model may be a neural network model, such as a Convolutional Neural Network (CNN), a Deep Neural Network (DNN), or the like. In some embodiments, the input of the stirring speed determination model may include the image difference, the first spacing, the second spacing, and the amount and/or the particle size of the xylitol crystal seeds added into the first crystallization kettle; and the output of the stirring speed determination model may include a newly determined stirring speed.

In some embodiments, the stirring speed determination model may be trained by a plurality of training samples with labels. Specifically, the plurality of training samples with labels may be input into an initial stirring speed determination model, the value of the loss function may be determined through the labels and results of the initial stirring speed determination model, and parameters of the initial stirring speed determination model may be iteratively updated based on the value of the loss function through gradient descent, or the like. Model training may be completed when a preset condition is satisfied, and a trained stirring speed determination model may be obtained. The preset condition may include that the loss function converges, the count of iterations reaches a threshold, etc.

In some embodiments, the training samples for training the stirring speed determination model may be obtained by collecting historical data of xylitol crystallization, selecting a portion of the historical data of which the actual crystallization quality satisfies the requirement, and slicing the portion of historical data that satisfies the requirement based on a predetermined time period (e.g., every 5 minutes) to obtain a plurality of reference historical time periods. Each of the plurality of reference historical time periods may include a historical image difference, a historical first interval, a historical second interval, and a historical amount and/or particle size of xylitol crystal seeds added into the first crystallization kettle. The labels may include the stirring speed of a next adjacent reference historical time period of each of the plurality of reference historical time periods.

The stirring speed determination model enables effective prediction of the stirring speed based on better historical crystallization data to determine the stirring speed that is more conducive to crystallization, thereby generating a more accurate frequency conversion instruction.

Operation 340, the frequency conversion instruction may be generated based on the stirring speed.

In some embodiments, the frequency conversion instruction may indicate a change in the stirring speed. For example, if the current stirring speed is 40 rpm and a new stirring speed determined in operation 330 is 25 rpm, the frequency conversion instruction may be a reduction of 15 rpm (or −15 rpm).

It should be noted that the foregoing description of process 200 and the operations 310-340 is for exemplification and illustration only, and does not limit the scope of application of the present disclosure. For those skilled in the art, various corrections and modification can be made to the process 200 and the operations 330-340 under the guidance of the present disclosure. However, these corrections and modifications remain within the scope of the present disclosure.

The effects of the preparation system and the preparation method for the xylitol crystals of the present disclosure are further illustrated below with reference to a plurality of Examples.

### Example 1

A preparation method for xylitol crystals of some embodiments of the present disclosure comprises the following operations.

Operation 11, xylitol hydrogenation solution was blended with dicrystalline sugar solution in a certain ratio, and then the mixed solution including the xylitol hydrogenation solution blended with the dicrystalline sugar solution sequentially passed through the decolorization tank 2 for decolorization treatment, the ion exchange column 3 for ion exchange treatment, a 400 Da nanofiltration system for treatment, and the first evaporator 5 for treatment to obtain xylitol concentrate solution with brix of 79%, temperature of 95° C., and conductivity of 1.958 μs/cm.

Operation 12, 10 tons of xylitol concentrate solution was fed into the first crystallization kettle 6 to be further concentrated at −0.095 MPa until the system temperature was cooled down to about 65° C. and 20 fine crystals were observed in the view window of the first crystallization kettle 6, then 0.0005% of xylitol crystal seeds were added to stimulate crystallization, at the same time, another 10 tons of xylitol concentrate solution was added to the first crystallization kettle 6. With a constant vacuum degree of −0.095 MPa and a constant temperature of 65 ° C., the solution in the first crystallization kettle 6 was stirred at an initial speed of 90 rpm with variable frequency and crystallized. After 7 h of crystallization, steam was fed to the first crystallization kettle to increase the system temperature to 67° C. and maintain for 0.5 h for crystallization to obtain xylitol sugar paste.

Operation 13, a separation treatment was performed on the obtained xylitol sugar paste by the first centrifuge 7 to obtain 8.7 tons of crystalline xylitol and 10.5 tons of primary mother liquor. The crystalline xylitol was dried by 80° C. hot air of the hot air drying tank 8 and dried by the cold air of the first fluidized bed dryer 9 to obtain xylitol crystal product. The primary mother liquor sequentially pass through the second evaporator 11 for concentration treatment, the second crystallization kettle 12 for crystallization treatment at the vacuum degree of −0.095 MPa and the temperature of 65° C., and the second centrifuge 13 for centrifugation treatment to obtain dicrystalline sugar and secondary mother liquor. The dicrystalline sugar was dried by the second fluidized bed dryer 14 and then entered the dicrystalline sugar dissolution tank 15 to be dissolved to obtain dicrystalline sugar solution. Then the dicrystalline sugar solution was delivered to the blending tank 1 to be blended and mixed with the xylitol hydrogenation solution. The secondary mother liquor sequentially passed through the third evaporator 17 for concentration treatment, the third crystallization kettle 18 for crystallization treatment, and the third centrifuge 19 for centrifugation treatment to obtain tricrystalline sugar and tertiary mother liquor. The tertiary mother liquor was stored in the liquid xylitol storage tank 22. The tricrystalline sugar was dried by the third fluidized bed dryer 20 and then entered the tricrystalline sugar dissolution tank 21 to be dissolved to obtain tricrystalline sugar solution. The tricrystalline sugar solution entered the primary mother liquor tank 10 through pipeline to be blended with the primary mother liquor for reuse.

The prepared xylitol crystal product was sieved to obtain a particle size distribution of the xylitol crystal product shown in Table 1.

Table 1 The particle size distribution of the xylitol crystal product prepared in Example 1

| | Particle size/mesh | | | | | |
|---|---|---|---|---|---|---|
| | >20 | 20-30 | 30-40 | 40-60 | 60-100 | <100 |
| Percentage/% | 19.17 | 60.38 | 14.19 | 3.95 | 2.26 | 0.05 |

The caking cycle of a small packaged sample of xylitol crystals prepared in Example 1 is 105 days.

### Example 2

Another preparation method for xylitol crystals of some embodiments of the present disclosure comprises the following operations.

Operation 21, xylitol hydrogenation solution was blended with dicrystalline sugar solution in a certain ratio, and then the mixed solution including the xylitol hydrogenation solution blended with the dicrystalline sugar solution sequentially pass through the decolorization tank 2 for decolorization treatment, the ion exchange column 3 for ion exchange treatment, the 400 Da nanofiltration system 4 for treatment, and the first evaporator 5 for treatment to obtain xylitol concentrate solution with brix of 80%, temperature of 95° C., and conductivity of 1.744 μs/cm.

Operation 22, 10 tons of xylitol concentrate solution was fed into the first crystallization kettle 6 to be further concentrated at −0.095 MPa until the system temperature was cooled down to about 65° C. and 25 fine crystals were observed in the view window of the first crystallization kettle 6, then 0.0005% of xylitol crystal seeds were added to stimulate crystallization, at the same time, another 10 tons of xylitol concentrate solution was added to the first crystallization kettle 6. With a constant vacuum degree of −0.095 MPa and the constant temperature of 65° C. of the system, the solution in the first crystallization kettle 6 was stirred at an initial speed of 90 rpm with variable frequency and crystallized. After 8 h of crystallization, steam was fed to the first crystallization kettle to increase the system temperature to 66.5° C. and maintain for 0.8 h for crystallization to obtain a xylitol sugar paste.

Operation 23, a separation treatment was performed on the obtained xylitol sugar paste by the first centrifuge 7 to obtain 8.6 tons of crystalline xylitol and 10.6 tons of primary mother liquor. The crystalline xylitol was dried by 80° C. hot air of the hot air drying tank 8 and dried by the cold air of the first fluidized bed dryer 9 to obtain xylitol crystal product. The primary mother liquor sequentially passed through the second evaporator 11 for concentration treatment, the second crystallization kettle 12 for crystallization treatment at the vacuum degree of −0.095 MPa and the temperature of 65° C., and the second centrifuge 13 for centrifugation treatment to obtain dicrystalline sugar and secondary mother liquor. The dicrystalline sugar was dried by the second fluidized bed dryer 14 and then entered the dicrystalline sugar dissolution tank 15 to be dissolved to obtain dicrystalline sugar solution. Then the dicrystalline sugar solution was delivered to the blending tank 1 to be blended and mixed with the xylitol hydrogenation solution. The secondary mother liquor sequentially passed through the third evaporator 17 for concentration treatment, the third crystallization kettle 18 for crystallization treatment, and the third centrifuge 19 for centrifugation treatment to obtain tricrystalline sugar and tertiary mother liquor. The tertiary mother liquor was stored in the liquid xylitol storage tank 22. The tricrystalline sugar was dried by the third fluidized bed dryer 20 and then entered the tricrystalline sugar dissolution tank 21 to be dissolved to obtain tricrystalline sugar solution. The tricrystalline sugar solution entered the primary mother liquor tank 10 through pipeline to be blended with the primary mother liquor for reuse.

The prepared xylitol crystal product was sieved to obtain a particle size distribution of the xylitol crystal product shown in Table 2.

Table 2 The particle size distribution of the xylitol crystal product prepared in Example 2

| | Particle size/mesh | | | | | |
|---|---|---|---|---|---|---|
| | >20 | 20-30 | 30-40 | 40-60 | 60-100 | <100 |
| Percentage/% | 19.28 | 58.31 | 16.11 | 3.40 | 2.57 | 0.33 |

The caking cycle of a small packaged sample of xylitol crystals prepared in Example 2 is 112 days.

To better describe the beneficial effects brought about by the present disclosure, the present disclosure is further illustrated with reference to the following Comparative Example.

### Comparative Example

The Comparative Example of the present disclosure comprises the following operations.

Operation 31, xylitol hydrogenation solution was blended with primary centrifugal mother liquor in a certain ratio, and then a decolorization treatment, ion exchange, and evaporation were performed, respectively, to obtain xylitol concentrate solution with a brix of 80.5%, temperature of 95° C., and conductivity of 2.063 μs/cm.

Operation 32, 7 tons of xylitol concentrate solution was fed into a crystallization tank to be further concentrated at −0.095 MPa until the system temperature was cooled down to about 65° C. and a large number (>50) of fine crystals were observed in the view window of the crystallization tank, then 0.0005% of xylitol crystal seeds were added to stimulate crystallization, at the same time, another 13 tons of xylitol concentrate solution was added to the crystallization tank. With a constant vacuum degree of −0.095 MPa and a constant temperature of 65° C. of the system, the solution in the crystallization tank was stirred at a constant speed of 90 rpm and crystallized for 8 h to obtain xylitol sugar paste.

Operation 33, the obtained xylitol sugar paste was separated by high-speed centrifuge to obtain 8.8 tons xylitol crystals and 10.2 tons primary centrifugal mother liquor. The xylitol crystals were dried by 88° C. hot air and dried by cold air to obtain xylitol product. The primary centrifugal mother liquor was directly returned to be blended with the xylitol hydrogenation solution.

The prepared xylitol crystal product was sieved to obtain a particle size distribution of the xylitol crystal product shown in Table 3.

Table 3 The particle size distribution of the xylitol crystal product prepared in Comparative Example

| | Particle size/mesh | | | | | |
|---|---|---|---|---|---|---|
| | 20 | 20-30 | 30-40 | 40-60 | 60-100 | 100 |
| Percentage/% | 6.47 | 50.21 | 19.42 | 15.19 | 8.04 | 0.67 |

The caking cycle of a small packaged sample of xylitol crystals prepared in Comparative Example is 45 days.

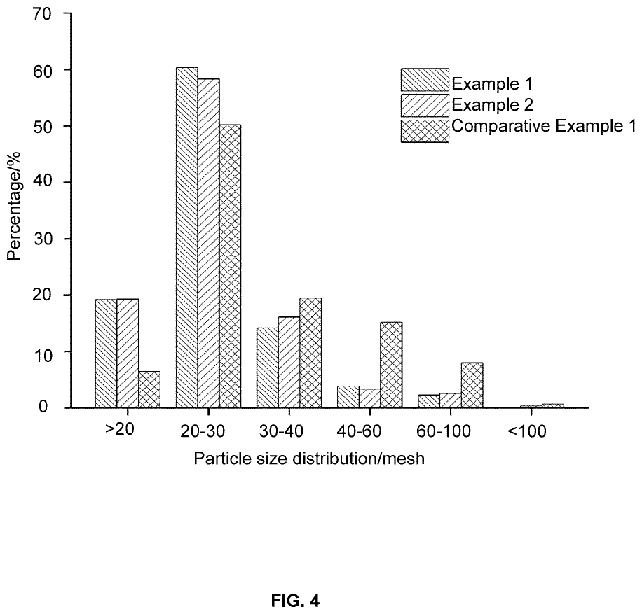

Compared with the Comparative Example, Example 1 and Example 2 obtained xylitol crystal products with larger particles and narrower particle size distributions by reducing returning of the xylitol mother liquor, adding the nanofiltration system to remove low-molecular-weight impurities, optimizing the crystallization process, adopting an alcoholic washing in the centrifugation process, etc. In addition, the caking cycle of xylitol crystal products was significantly improved. The comparison data is shown in Table 4 and FIG. 4.

Table 4 Comparison of xylitol crystal product prepared in each of Examples with xylitol crystal product prepared in Comparative Example

| | Percentage of particle | Caking cycle days of |
|---|---|---|
| Item | size >30 mesh/% | small packaged sample/day |
| Example 1 | 79.55 | 105 |
| Example 2 | 77.59 | 112 |
| Comparative | 56.68 | 45 |
| Example | | |

It can be seen from the above table that in the comparison item of the percentage of particle size >30 mesh, the xylitol crystal product prepared in Example 1 is 40% higher than that of Comparative Example, and the xylitol crystal product prepared in Example 2 is 36% higher than that of the Comparative Example. In addition, the caking cycles of the xylitol crystal products prepared in Example 1 and Example 2 are significantly higher than that of Comparative Example.

The basic concept has been described above. Obviously, for those skilled in the art, the above detailed disclosure is only an example, and does not constitute a limitation to the present disclosure. Although not expressly stated here, those skilled in the art may make various modifications, improvements and corrections to the present disclosure. Such modifications, improvements and corrections are suggested in this disclosure, so such modifications, improvements and corrections still belong to the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures, or characteristics in one or more embodiments of the present disclosure may be properly combined.

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed by way of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in this disclosure and help the understanding of one or more embodiments of the invention, in the foregoing description of the embodiments of the present disclosure, sometimes multiple features are combined into one embodiment, drawings or descriptions thereof. This method of disclosure does not, however, imply that the subject matter of the disclosure requires more features than are recited in the claims. Rather, claimed subject matter may lie in less than all features of a single foregoing disclosed embodiment.

In some embodiments, counts describing the quantity of components and attributes are used. It should be understood that such counts used in the description of the embodiments use the modifiers “about”, “approximately” or “substantially” in some examples. Unless otherwise stated, “about”, “approximately” or “substantially” indicates that the stated figure allows for a variation of ±20%. Accordingly, in some embodiments, the numerical parameters used in the disclosure and claims are approximations that can vary depending upon the desired characteristics of individual embodiments. In some embodiments, numerical parameters should consider the specified significant digits and adopt the general digit retention method. Although the numerical ranges and parameters used in some embodiments of the present disclosure to confirm the breadth of the range are approximations, in specific embodiments, such numerical values are set as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A preparation system for xylitol crystals, comprising:
a blending tank, a decolorization tank, an ion exchange column, a nanofiltration system, a first evaporator, a first crystallization kettle, a first centrifuge, a hot air drying tank, and a first fluidized bed dryer that are sequentially connected through pipelines;
a primary mother liquor storage tank, a second evaporator, a second crystallization kettle, a second centrifuge, a second fluidized bed dryer, and a dicrystalline sugar dissolution tank that are sequentially connected through pipelines; and
a secondary mother liquor storage tank, a third evaporator, a third crystallization kettle, a third centrifuge, a third fluidized bed dryer, and a tricrystalline sugar dissolution tank that are sequentially connected through pipelines, wherein
a solid outlet of the first centrifuge is connected with an inlet of the hot air drying tank through pipeline, a liquid outlet of the first centrifuge is connected with an inlet of the primary mother liquor storage tank through pipeline, a solid outlet of the second centrifuge is connected with an inlet of the second fluidized bed dryer through pipeline, a liquid outlet of the second centrifuge is connected with an inlet of the secondary mother liquor storage tank through pipeline, the dicrystalline sugar dissolution tank is provided with a water inlet for pure water, an outlet of the dicrystalline sugar dissolution tank is connected with an inlet of the blending tank, the tricrystalline sugar dissolution tank is provided with a water inlet for pure water, an outlet of the tricrystalline sugar dissolution tank is connected with the inlet of the primary mother liquor storage tank through pipeline, the blending tank is provided with an inlet for a raw material of xylitol hydrogenation solution, the blending tank is configured to mix the raw material of xylitol hydrogenation solution with dicrystalline sugar solution delivered from the dicrystalline sugar dissolution tank, and an output of an outlet of the first fluidized bed dryer is prepared xylitol crystals.

2. The preparation system of claim 1, further comprising a liquid xylitol storage tank, wherein an outlet of the third centrifuge is connected with an inlet of the liquid xylitol storage tank through pipeline.

3. The preparation system of claim 1, further comprising an image sensor and a microprocessor, wherein the microprocessor is configured to:
generate a frequency conversion instruction and send the frequency conversion instruction to the first crystallization kettle to regulate a stirring speed of the first crystallization kettle; and,
generate a cycle acquisition instruction and send the cycle acquisition instruction to the image sensor, the image sensor being located near a view window of the first crystallization kettle and configured to acquire solution images of xylitol concentrate solution.

4. The preparation system of claim 3, wherein a size of the view window of the first crystallization kettle is 1 cm², the image sensor includes a 10×10 magnification microscope, and the microscope is configured to observe a count of particles of the xylitol crystals through the view window.

5. The preparation system of claim 3, wherein the microprocessor is configured to:
obtain, based on an acquisition cycle of the cycle acquisition instruction, a solution image of current xylitol concentrate solution from the image sensor;
determine, based on the solution image of the current xylitol concentrate solution, an image difference;
determine the stirring speed based on the image difference, a first interval, a second interval, and an amount and/or a particle size of xylitol crystal seeds added into the first crystallization kettle, wherein the first interval is a time difference between a time when the xylitol concentrate solution enters the first crystallization kettle and a time when the xylitol crystal seeds are added, and the second interval is a time difference between the time when the xylitol crystal seeds are added and a current time; and
generate, based on the stirring speed, the frequency conversion instruction.

6. The preparation system of claim 5, wherein the microprocessor is configured to:
determine the stirring speed based on the image difference, the first interval, the second interval, and the amount and/or the particle size of the xylitol crystal seeds added into the first crystallization kettle through a stirring speed determination model, the stirring speed determination model being a machine learning model.

7. A preparation method for xylitol crystals, performed using the preparation system for the xylitol crystals of claim 1, wherein the preparation method comprises:
operation 210, in the blending tank, blending the raw material of xylitol hydrogenation solution with the dicrystalline sugar solution in a certain ratio, and then sequentially passing mixed solution including the raw material of xylitol hydrogenation solution blended with the dicrystalline sugar solution through the decolorization tank for decolorization treatment, the ion exchange column for impurity removal treatment, the nanofiltration system for filtration treatment, and the first evaporator for evaporation and concentration treatment to obtain xylitol concentrate solution, wherein brix of the xylitol concentrate solution is within a range of 78-82%, temperature of the xylitol concentrate solution is within a range of 90-100° C., and conductivity of the xylitol concentrate solution is smaller than 20 μs/cm;
operation 220, entering the xylitol concentrate solution into the first crystallization kettle through pipeline, including: feeding the first crystallization kettle with a first volume of xylitol concentrate solution, controlling a vacuum degree of the first crystallization kettle to be within a range from −0.095 MPa to −0.098 MPa and temperature of the first crystallization kettle to be within a range from 64° C. to 68° C., and adding xylitol crystal seeds until xylitol crystals begin to crystalize in the first crystallization kettle and a count of particles of the xylitol crystals observed from a view window of the first crystallization kettle is within a preset range, adding a second volume of xylitol concentrate solution to the first crystallization kettle, performing a variable frequency stirring for 7-12 h, and at an end of crystallization, feeding steam to increase system temperature by 1-2° C. and maintaining for 0.5-2.0 h to obtain a xylitol sugar paste, wherein a ratio of the first volume to the second volume is within a range of 1:0.8-1.5;
operation 230, performing, using the first centrifuge to separate the xylitol sugar paste obtained in operation 220 to obtain crystalline xylitol and primary mother liquor, entering the primary mother liquor into the primary mother liquor tank through pipeline for temporary storage, and passing the crystalline xylitol through the hot air drying tank for drying treatment and the first fluidized bed dryer for cold air drying treatment to obtain the prepared xylitol crystals; sequentially passing the primary mother liquor through the second evaporator for concentration treatment, the second crystallization kettle for crystallization treatment, and the second centrifuge for centrifugation treatment to obtain dicrystalline sugar and secondary mother liquor, and entering the secondary mother liquor into the secondary mother liquor tank through pipeline for temporary storage; passing the dicrystalline sugar through the second fluidized bed dryer for drying treatment and the dicrystalline sugar dissolution tank for dissolution treatment, inputting the pure water to the dicrystalline sugar dissolution tank to dissolve the dicrystalline sugar to obtain dicrystalline sugar solution, and entering the dicrystalline sugar solution into the blending tank through pipeline for blending; and
operation 240, sequentially passing the secondary mother liquor obtained in operation 230 through the third evaporator for concentration treatment, the third crystallization kettle for crystallization treatment, and the third centrifuge for centrifugation treatment to obtain tricrystalline sugar and tertiary mother liquor, passing the tricrystalline sugar through the third fluidized bed dryer for drying treatment and the tricrystalline sugar dissolution tank for dissolution treatment, inputting the pure water to the tricrystalline sugar dissolution tank to dissolve the tricrystalline sugar to obtain tricrystalline sugar solution, and entering the tricrystalline sugar solution into the primary mother liquor tank through pipeline to blend the tricrystalline sugar solution with the primary mother liquor, the tertiary mother liquor being stored as liquid xylitol.

8. The preparation method of claim 7, wherein in operation 210, a molecular weight cut-off of a nanofiltration membrane of the nanofiltration system is within a range of 300-800 Da.

9. The preparation method of claim 7, wherein in operation 220, the first crystallization kettle is a vacuum evaporation crystallization kettle, an amount of the xylitol crystal seeds accounts 0.0004-0.0008% of a mass of solution in the first crystallization kettle, and a particle size of the xylitol crystal seeds is within a range of 80-100 mesh.

10. The preparation method of claim 7, wherein in operation 230, the xylitol sugar paste is sprayed and washed for 5-10 s using 80-100% concentration ethanol solution after the separation treatment is performed on the xylitol sugar paste using the first centrifuge.

11. The preparation method of claim 7, wherein in operation 230, temperature of hot air of the hot air drying tank is within a range of 75-85° C.

12. The preparation method of claim 7, wherein in operation 220, in the variable frequency stirring, a stirring speed gradually reduces from 90 rpm to 10 rpm.
